(19) United States
(12) Patent Application Publication
(10) Pub. No.: US 2023/0091193 A1
(43) Pub. Date: Mar. 23, 2023

(54) Methods and Systems for Detoxification and Cleansing of the Lymphatic and Blood Circulation Systems Using Epidermis Cupping and Quantum Means

(71) Applicant: Wei Chang, San Diego, CA (US)

(72) Inventor: Wei Chang, San Diego, CA (US)

(21) Appl. No.: 17/481,315

(22) Filed: Sep. 22, 2021

(51) Int. Cl. A61H 39/04

(52) U.S. Cl. CPC A61H 39/04; A61H 2201/013

(57) ABSTRACT

A traditional therapeutic method and system is provided for the detoxification and cleansing of the lymphatic and blood circulation systems of a human body using epidermis cupping and ancient herbal scented pastes, which is based on anatomical models and structures trained for healthcare therapy practitioners using the traditional Meridian theory to wholistically demonstrate that a living being with a flow of quantum energy defined as Chi, where breath can control Chi, Chi can command blood flow, and blood flow can nourish and feed, to integrate with modern quantum physics theory by demonstrating that the fundamental structure at the core of all life are forces of energy that are assembled or devoid of mass, which quantum strength of these forces can either hold clusters of particles together as a mass or disperse a mass into particles, where the aromatic quantum smell of herbal pastes play an important role in human medicines.

### DEFINITIONS

AP: stands for Acupressure or Acupuncture Point.

Chi: defined as a type of quantum energy flowing in a body that can be generated by various means including, but not limited to, mechanically, electrically, magnetically, biologically, biochemically, radioactively, biophysically, aromatically, heat, consciously, unconsciously, subconsciously, independently, dependently, actively, passively, mentally, physically, or their combinations.

Cun: defined in Traditional Chinese Medicine as a distance unit to provide proportional measurements on the human body.

MG: stands for Meridian Gate, belong to a subset of APs that can be enabled at, near or around with Chi or scented herbs or acupuncture or moxibustion or other means to effect or control certain Meridian channels.

Meridian or Meridian channel: defined as a quantum pathway for the quantum energy Chi to flow in a body through collections of APs, corresponding to functional efficacies of various internal organs associated with numerous factors including, but not limited to, time, seasons, yin, yang, active or passive actions, physical or emotional characteristics related to Chi.

### BACKGROUND OF THE INVENTION

The traditional Meridian theory has been researched, studied and practiced by healthcare practitioners worldwide for centuries using both ancient and modern anatomical teaching and practical training tools to illustrate that a human body encompasses a total twenty-six core Meridians or Meridian channels with a total six hundred seventy (670) APs as shown in FIG. 1 and FIG. 2.

With an anatomical sagittal/median plane, there are twelve core Meridian channels in both left and right half of a human body. Each consists of Lung Meridian (LU) with eleven (11) APs as shown in FIG. 3, Large Intestine Meridian (LI) with twenty (20) APs as shown in FIG. 4, Stomach Meridian (ST) with forty-five (45) APs as shown in FIG. 5, Spleen Meridian (SP) with twenty-one (21) APs as shown in FIG. 6, Heart Meridian (HT) with nine (9) APs as shown in FIG. 7, Small Intestine Meridian (SI) with nineteen (19) APs as shown in FIG. 8, Bladder Meridian (BL) with sixty-seven (67) APs as shown in FIG. 9, Kidney Meridian (KD/KID) with twenty-seven (27) APs as shown in FIG. 10, Pericardium Meridian (PC/PE) with nine (9) APs as shown in FIG. 11, Sanjiao Meridian (SJ/TW) with twenty three (23) APs as shown in FIG. 12, Gallbladder Meridian (GB) with forty-four (44) APs as shown in FIG. 13, and Liver Meridian (LV/LIV) with fourteen (14) APs as shown in FIG. 14.

With an anatomical frontal/coronal plane, there are two core Meridians embodied from the groin up where Conception Vessel (CV/REN) residing in the center front with twenty-four (24) APs as shown in FIG. 15 and Governing Vessel (GV/DU) circling the center back to top and front with twenty-eight (28) APs as shown in FIG. 16.

With empirical studies and practices using the Asian Meridian theory, a healthcare practitioner can perform certain therapeutic massage techniques to enact and transform Chi to excite and enable the energy flow between or among certain Meridian channels at, near or around certain MPs. Conversely, the practitioner can also perform certain therapeutic massage techniques at, near or around certain MPs to slow or reduce or stop the energy flow between or among certain Meridian channels.

With modern anatomical theory, the two basic postnatal routes for blood flow inside a living human being are the systemic circulation and the pulmonary circulation as shown in FIG. 17, where the systemic circulation includes all arteries and arterioles that carry oxygenated blood from the left ventricle to systemic capillaries, plus the veins and venules that carry deoxygenated blood returning to right atrium after flowing through body organs, which all functions are controlled by pulmonary circulation due to breathing. The lymphatic system is the most important immune response system in a human body that contains sites of maturation and proliferation of B cells and T cells to protect against all disease-causing microbes and viruses as shown in FIG. 18, which it drains excess interstitial fluid from tissue spaces and returns proteins and fluid to blood; and transports dietary lipids from gastrointestinal tract to blood. Whereas, toxic wastes of a human body are also carried in the lymphatic and blood vessel circulation systems. All waste and nutrient interchanges are carried out in lymphatic capillaries and blood capillaries circulations as shown in FIG. 19.

By integrating ancient Meridian, modern anatomy and quantum physics theories, it is claimed and demonstratable by this invention disclosure that a new way of understanding and practices to embolden and enhance the immune response system of a human body can be realized, for which a new method and system can be implemented to detox and cleanse the lymphatic and blood vessel circulation systems by transforming Chi at, near or around the MGs to extract the waste substances out of human cesspools of vessels and sites through lymphatic and blood capillaries by applying the negative energy force or means through epidermis of a human integumentary system.

The cupping practice of a traditional massage therapy is a typical example of applying the negative energy force to augment or enhance the circulation systems of a human body as shown in FIG. 20, whereas the ancient oxygen burning cupping is applied on top picture, and the modern vacuum cupping is applied on bottom picture.

To facilitate the detoxification and cleansing of the lymphatic and blood circulation systems, it is natural and preferable to pre-treat the extractable substances of a human body first by making them freely flowing, easy to separate or decompose, away from their clustering mass, reducing or breaking clot mass into particles, agitated into free ions. Basing on modern quantum physic theory, the bonding forces of energy between and among clustered masses can be weakened and reduced by applying the proper quantum energy to force through the quantum conduits or tunnels of MGs. It is demonstrable by this invention disclosure that a set of proprietary aromatic herbal scent unguent or ointment pastes can be applied at, near or around a set of proprietary MGs can act as the quantum energy to reduce and break the bonding force of energy among clustered masses with high efficacy, while different aromatic herbal scent pastes will have different impacts and efficacies as shown in FIG. 21. Thus, different forms of natural or chemical intakes by a human body will have different quantum effects to the bonding forces of energy with different efficacies for the treatment.

With the above knowledge and theories, the present invention disclosure shall illustrate that a method and system with preferred proprietary sets of therapeutic AP and MG massages, epidermis cupping and quantum aromatic pasting combinations, the detoxification and cleansing of lymphatic and blood circulation systems can be realized and protected effectively and quantitatively. The immune system of a human body can be recalled, revitalized and regenerated.

### BRIEF SUMMARY OF THE INVENTION

The present invention disclosure illustrates, by applying a set of preferred proprietary traditional massage therapy procedures, techniques, methods, and processes including, but not limiting to, proprietary sets of ancient acupressure therapy techniques, sets of proprietary quantum aromatic herbal scent pastes, sets of traditional cupping therapies, consecutive sets of common vacuum cupping therapies, the detoxification and cleansing of the lymphatic and blood circulation systems can be realized and achieved with following details:
1. By applying preferred pre-determined proprietary therapeutic ancient massage techniques at, near or around certain APs and MPs for an optimized duration can excite, energize, open, transform, and transport Chi energy to effect and control certain preferred Meridian channels;
2. By performing sets of professional trained traditional cupping massage session at, near or around preferred proprietary APs with an optimized duration can further enhance the functional efficacies of Chi on certain Meridian channels;
3. By applying sets of certain aromatic herbal scent ointment pastes at, near or around sets of proprietary MGs respectively for an optimized duration after completing procedure 2 can affect and open certain preferred Meridian channels to enable and transport quantum aromatic herbal scent energy permeating deeply into associated lymphatic and blood capillaries circulation systems;
4. Within an optimal duration of completing procedure 3, sets of professional trained common vacuum cupping therapy sessions with preferred negative vacuum pressures will be applied, adjusted, and maintained in standard atm pressure at, near or around sets of proprietary Meridian APs for an optimal period at first, then increase the vacuum pressure by empirical preferred standard atm pressure to maintain a stable negative pressure for another optimal period to end the session, while maintaining a clinical therapy hygiene standard practice to treat and dispose the substance sucked out through epithelial tissue caused by the vacuum cuppings, then continue to perform the same therapeutic vacuum cupping session two more times in the same day with the same optimized duration for each session, whereas each consecutive session shall begin a minimum period separating from the previous session to guarantee a sufficient time to reset or re-balance the homeostasis of a human body;
5. To continuously perform the same vacuum cupping therapy session described in procedure 4 above three (3) times a day consecutively total to seven (7) days in a row, whereas taking out every vacuum cup having no substance sucked out through the epithelial tissues during each session;
6. Then by continuously performing the similar vacuum cupping therapy session as described in procedures 4 and 5 with a new set of reduced negative pressures three (3) times a day consecutively for another total 5 days in a row, whereas the reduced negative pressure session shall start at new optimal negative pressure maintained for a preferred period first, then increase to a new preferred negative pressure for another optimal period to end the session, while constantly maintaining a clinical standard therapy hygiene practice to treat and dispose all epidermal substances sucked out during the therapy sessions;
7. Then to continuously perform the similar vacuum cupping therapy sessions described in procedure 6 with a new set of reduced negative pressures three (3) times a day consecutively until no empty cups remain, whereas the entire vacuum cupping therapy session will then be completed, whereas a clinical standard therapy hygiene practice is strictly maintained to treat and dispose all epidermal substances sucked out during the entire therapy sessions;
8. Then by applying a set of quantum aromatic herbal scent pastes at, near or around a set of proprietary MGs to heal and close the preferred Meridian channels for a maximum duration of four (4) hours from 2 hours up to 24 hours after the completion of procedure 7, the entire treatment is completed;
9. In order to ensure, guarantee and maintain high quality and standards for all procedural methods and performances, all healthcare therapy practitioners shall be clinically trained and re-certified with highly standardized professional techniques and hygiene practices as an epidermal detoxification and cleansing professional, whereas all therapeutic tools and means used in the procedures, practices and processes are commonly available without infringing upon any patent to any third party;
10. The preferred embodiments, procedure, process, method, and performance techniques summarized here shall not be construed as limiting;
11. Additions and modifications to what is expressly described here are included within the scope of the invention.

### BRIEF OF DESCRIPTION OF THE DRAWINGS

To better understand the present invention of methods and systems for the detoxification and cleansing of the lymphatic and blood vessel systems, it is best to read the foregoing summary, the following detailed description of the preferred embodiment, in conjunction with the descriptions of appended drawings below, in which:

FIG. 1 is an orthographic front view of the embodiment that left showing the main APs in dots of core Meridians of a human-anterior body, and right showing a human-anterior anatomy guide of the skeletal muscle system.

FIG. 2 is an orthographic front view of the embodiment that right showing all APs in dots of core Meridians of a human-posterior body, and left showing a human-posterior anatomy guide of the skeletal muscle system.

FIG. 3 is a perspective view of the embodiment that right showing all eleven (11) APs of Lung Meridian/Channel (LU) of a human-anterior body, while left showing a human-anterior anatomy relating to Chi movement of core Meridians.

FIG. 4 is a perspective view of the embodiment that right anterior and left posterior showing all twenty (20) APs of Large Intestine Meridian (LI), while left anterior and right posterior showing a human anatomy relating to Chi movement of core Meridians.

FIG. 5 is a perspective view of the embodiment that completely showing all forty-five (45) APs of Stomach Meridian (ST), while also showing a human anatomy relating to Chi movement of core Meridians.

FIG. 6 is a perspective view of the embodiment that completely showing all twenty-one (21) APs of Spleen Meridian (SP), while also showing a human anatomy relating to Chi movement of core Meridians.

FIG. 7 is a perspective view of the embodiment that completely showing all nine (9) APs of Heart Meridian (HT), while also showing a human anatomy relating to Chi movement of core Meridians.

FIG. 8 is a perspective view of the embodiment that completely showing all nineteen (19) APs of Small Intestine Meridian (SI), while also showing a human anatomy relating to Chi movement of core Meridians.

FIG. 9 is a perspective view of the embodiment that completely showing all sixty-seven (67) APs of Bladder Meridian (BL), while also showing a human anatomy relating to Chi movement of core Meridians.

FIG. 10 is a perspective view of the embodiment that completely showing all twenty-seven (27) APs of Kidney Meridian (KID/KD), while also showing a human anatomy relating to Chi movement of core Meridians.

FIG. 11 is a perspective view of the embodiment that completely showing all nine (9) APs of Pericardium Meridian (PC/PE), while also showing a human anatomy relating to Chi movement of core Meridians.

FIG. 12 is a perspective view of the embodiment that completely showing all twenty-three (23) APs of Sanjiao Meridian (SJ/TW), while also showing a human anatomy relating to Chi movement of core Meridians.

FIG. 13 is a perspective view of the embodiment that completely showing all forty-four (44) APs of Gallbladder Meridian (GB), while also showing a human anatomy relating to Chi movement of core Meridians.

FIG. 14 is a perspective view of the embodiment that completely showing all fourteen (14) APs of Liver Meridian (GB), while also showing a human anatomy relating to Chi movement of core Meridians.

FIG. 15 is a perspective view of the embodiment that completely showing all twenty-four (24) APs of REN Meridian (REN/CV), while also showing a human anatomy relating to Chi movement of core Meridians.

FIG. 16 is a perspective view of the embodiment that completely showing all twenty-eight (28) APs of DU Meridian (DU/GV), while also showing a human anatomy relating to Chi movement of core Meridians.

FIG. 17 is a schematic diagram showing the systemic circulation of a human body controlled by breath, and using arrows to indicate the direction of blood flow, and to illustrate how the circulation nourishes and feeds.

FIG. 18 is an anterior view of the principal components of lymphatic system, which consists of lymph, lymphatic vessels, lymphatic tissues, and red bone marrow to provide main functions of draining excess interstitial fluids; transporting dietary lipids from the gastrointestinal tract to the blood; and protecting against invasion through immune responses.

FIG. 19 is a schematic diagram showing the relationship of the lymphatic circulation system to the cardiovascular circulation system using arrows to indicate the direction of flow of lymph and blood in circulation, and to illustrate how the nutrients, fluid, and other lipids are exchanged in capillaries circulation systems.

FIG. 20 is showing the therapeutic practice of applying the traditional cupping therapy on top picture, and applying the common vacuum cupping therapy on bottom picture.

FIG. 21 is showing on top picture to paste the traditional herbal ointments by a therapist, and to self-paste in the bottom picture.

FIG. 22 is a summary of perspective views for the proportional measurements on the human body using Cun as defined in Traditional Chinese Medicine.

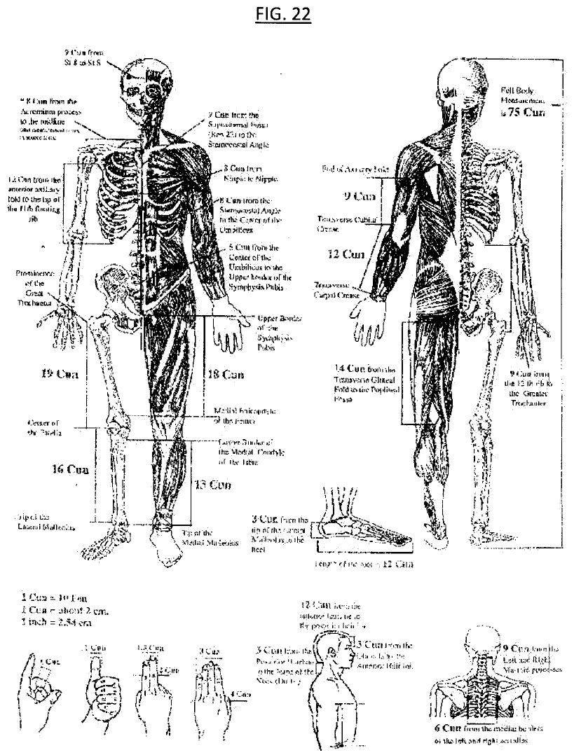

### DETAIL DESCRIPTION OF THE PREFERRED EMBODIMENT

In accordance with the present invention, the preferred embodiments described here should not be construed limiting for the purpose of detoxification and cleansing of the lymphatic and blood circulation systems. Additions and modifications to what is expressly described here are included within the scope of the invention. The features of the various of embodiments described here are not mutually exclusive and can exist in various combinations and permutations, even if such combinations or permutations are not made express here, without departing from the spirit and scope of the invention.

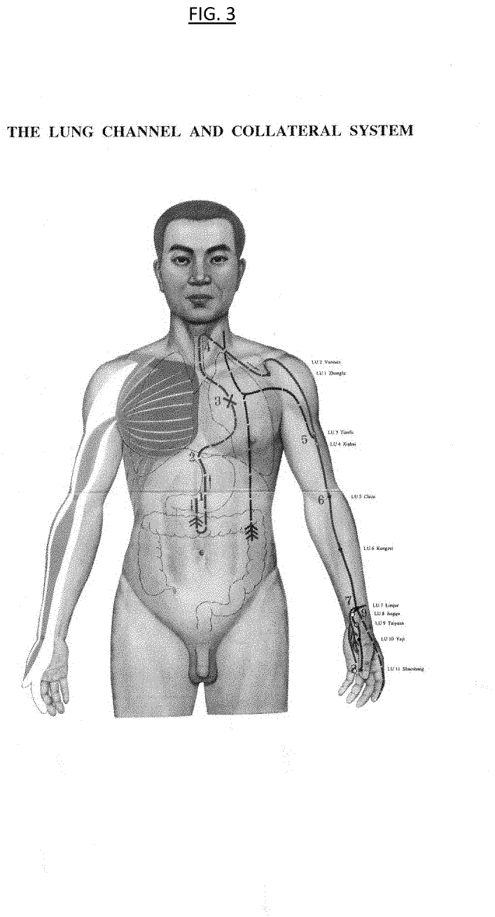

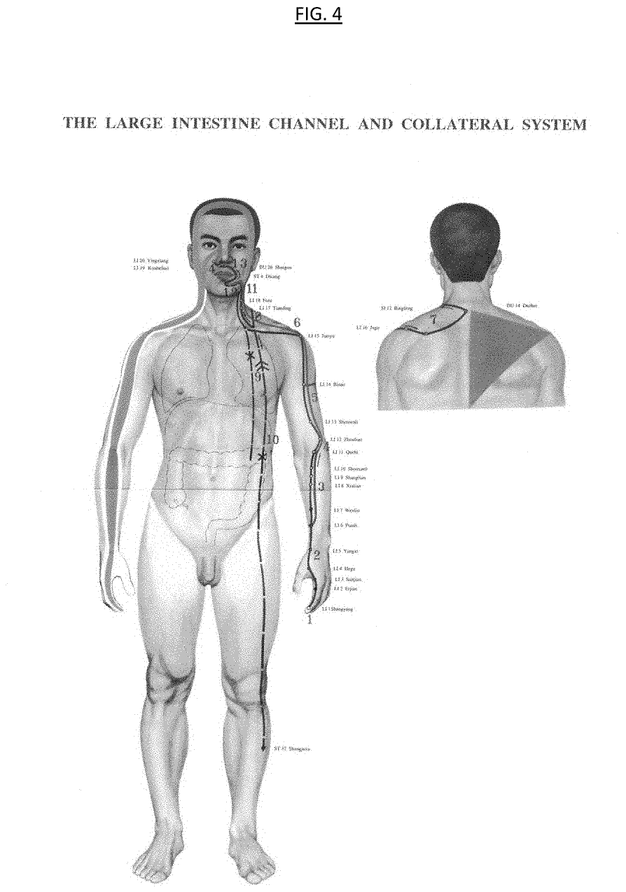

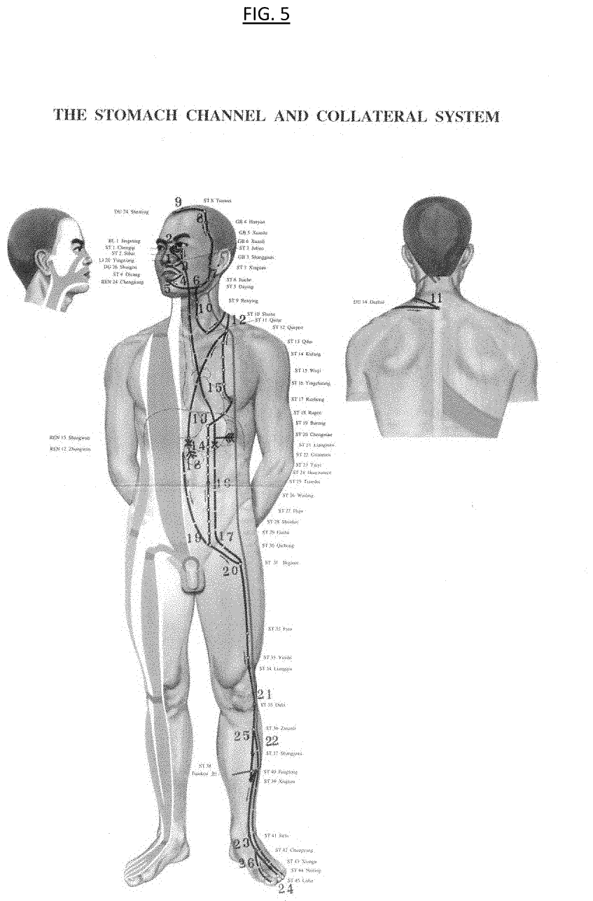

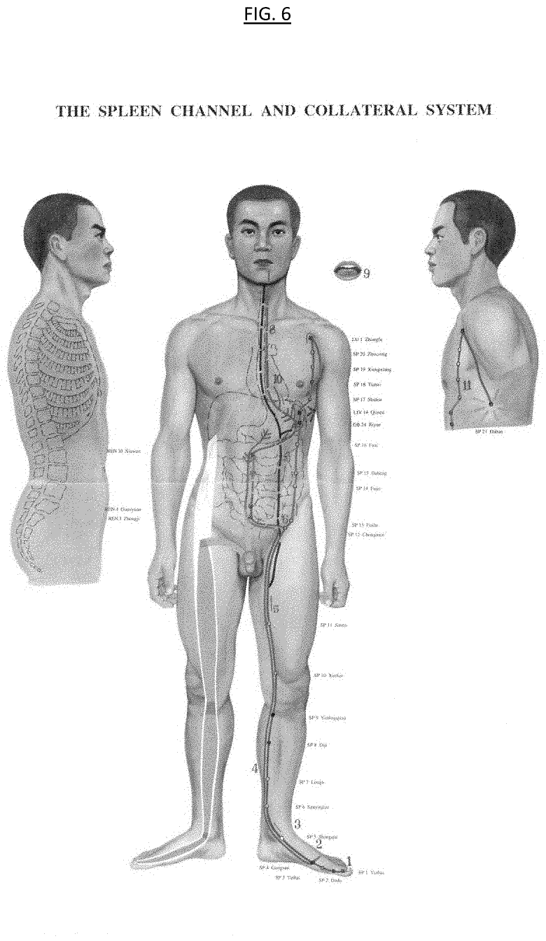

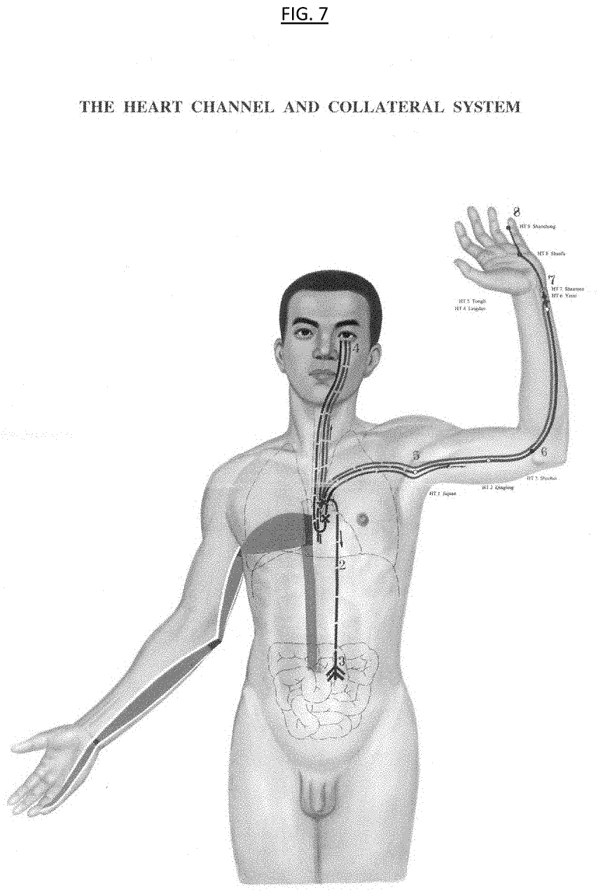

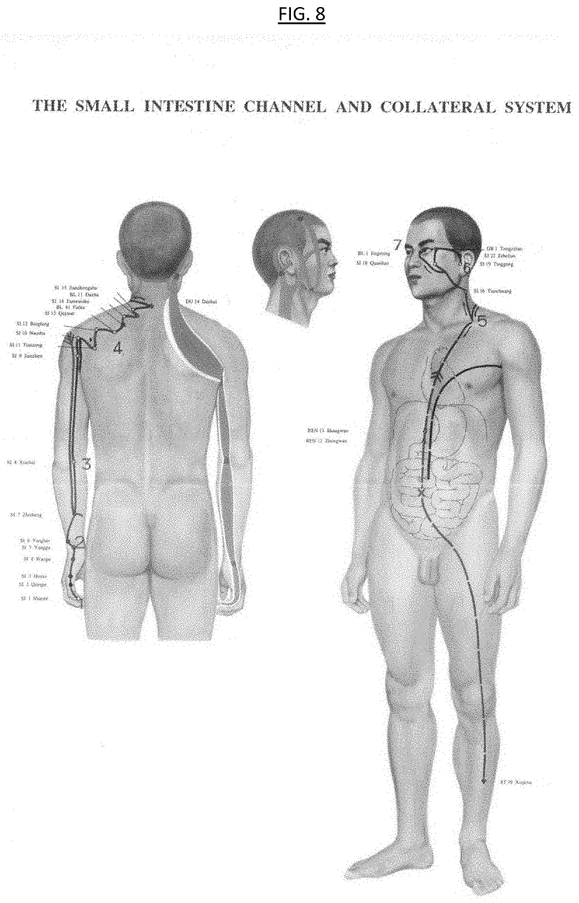

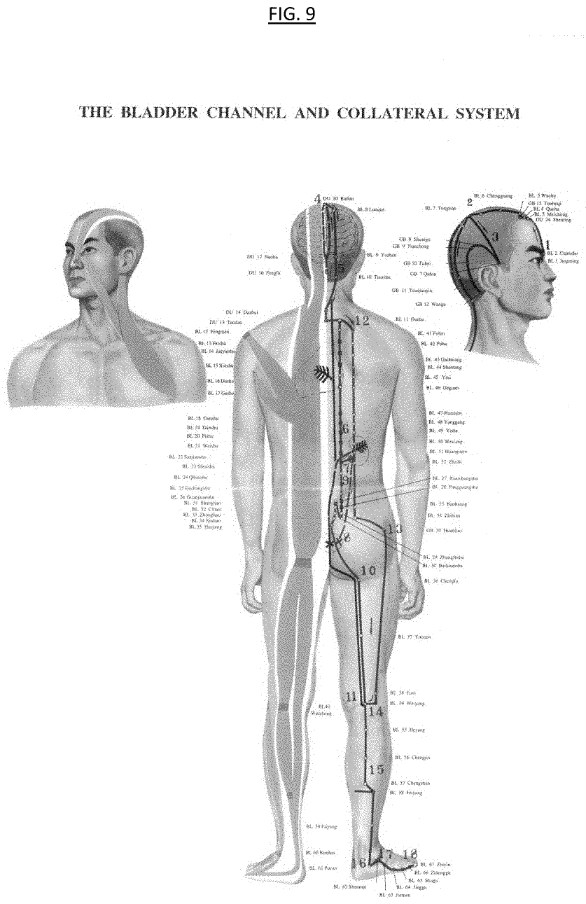

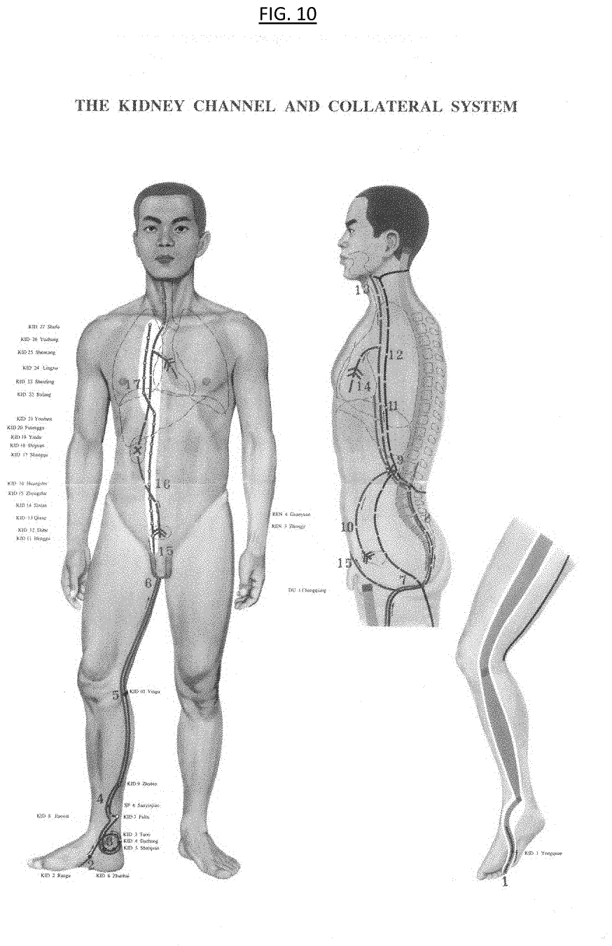

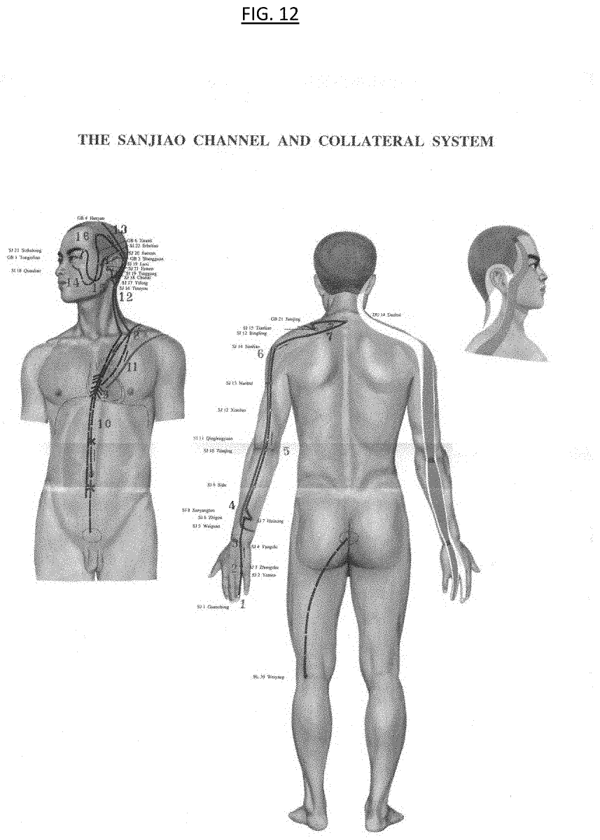

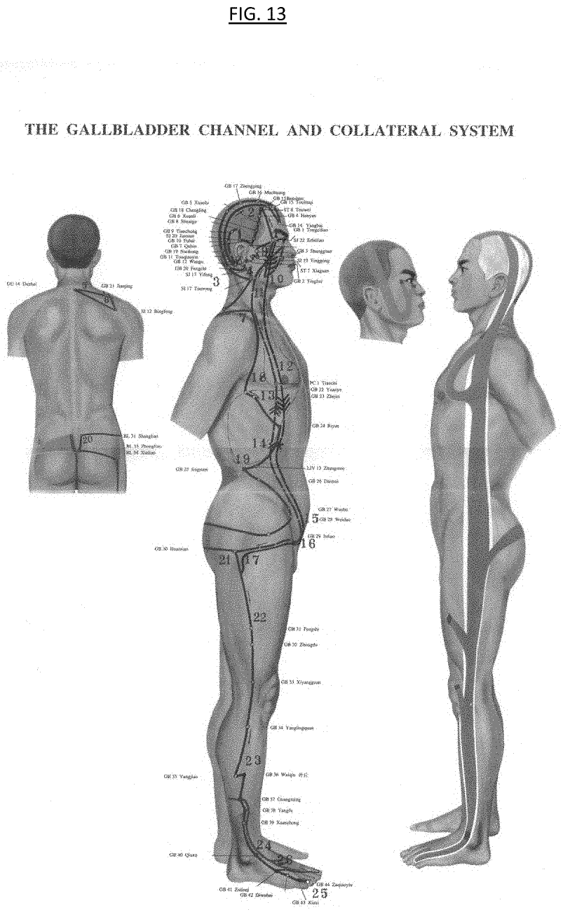

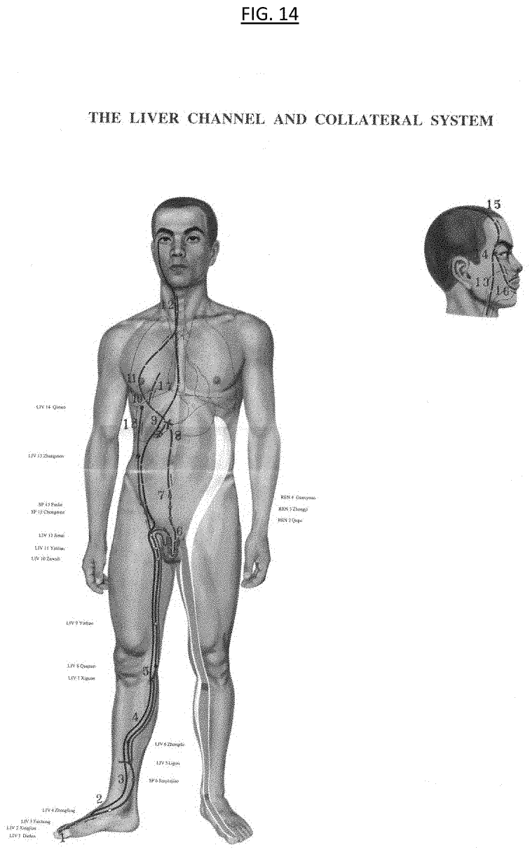

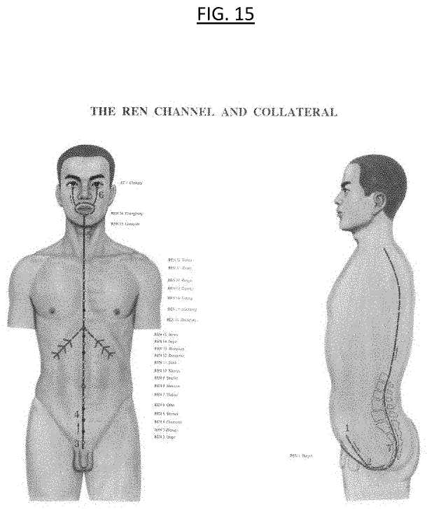

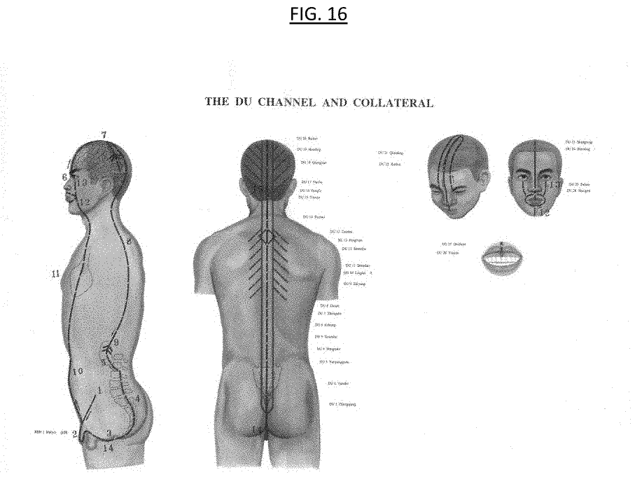

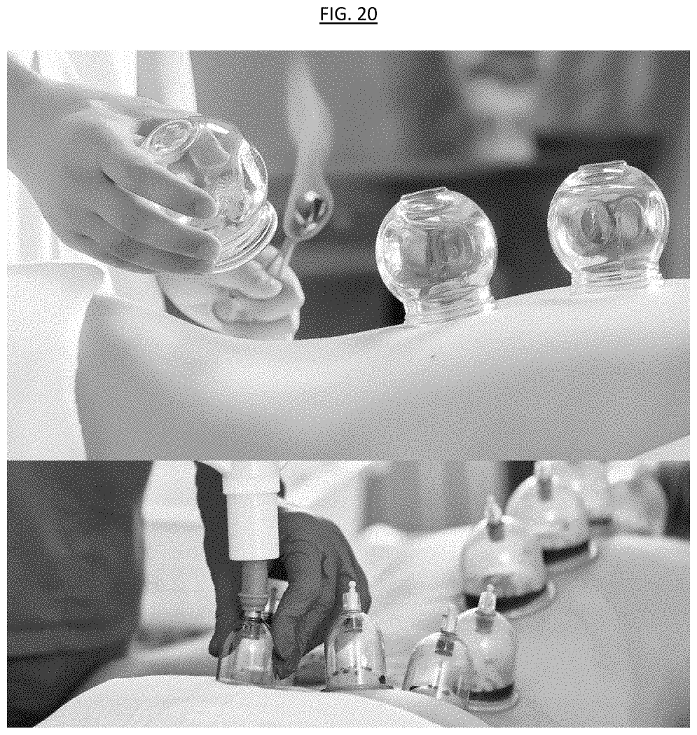

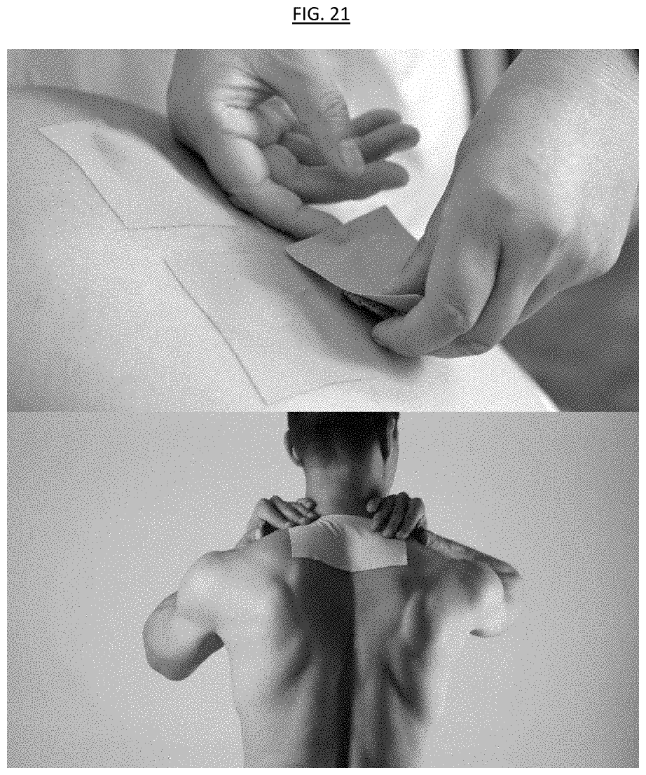

The preferred embodiment below is a practical example therapy treatment with methods and systems that can be implemented and realized to detox and cleanse the lymphatic and blood circulation systems of a human body using epidermis cupping and quantum means, whereas:
1. A set of Tui, Na, An, Tien and Pok acupressure massage techniques will be applied systematically, professionally, and effectively at, near and around the following APs in Meridians of LU1 to LU11 in FIG. 3, LI16 in FIG. 4, ST19 to ST27 in FIG. 5, SP14 to SP16 in FIG. 6, SI10 to SI15 in FIG. 8, BL11 to BL35 and BL41 to BL54 in FIG. 9, KID/KD13 to KID/KD21 in FIG. 10, SJ14 and SJ15 in FIG. 12, GB24 and GB25 in FIG. 13, REN/CV6 to REN/CV22 in FIG. 15, and DU2 to DU20 in FIG. 16 for an optimal 10 minutes;
2. Then to consecutively concentrate the acupressure massage on MPs of DU/GV4, DU/GV14, REN/CV8, REN/CV17, both left and right ST35, BL25, SI11, and LIV/LV12 for an optimal 10 minutes;
3. Then to immediately apply the traditional cupping therapy as shown in FIG. 20 epidermically at, near or around the following preferred fourteen (14) APs: SI11×2 as shown in FIG. 8, BL13×2, BL15×2, BL18×2, BL20×2, and BL23×2 as shown in FIG. 9, DU/GV3 and DU/GV14 as shown in FIG. 16 for an optimal 10 minutes;
4. Then to immediately apply two sets of preferred aromatic herbal scent pastes as shown in FIG. 21, whereas one set is applied at, near or around MGs SI11 and LIV/LV12 for energizing and opening the preferred Meridian channels in procedure 1 above; and whereas another set is applied at, near or around MGs DU/GV14, REN/CV8, BL25, REN/CV17, and ST35 for transforming and transporting Chi to preferred Meridian channels in procedure 1 above from minimum three (3) hours up to maximum 24 hours;
5. Then to start the first batch of vacuum cupping therapy sessions three (3) times a day consecutively for 7 days with following procedures to be applied by a healthcare therapy practitioner:
5.1. a set of professional trained vacuum cupping therapy sessions as shown in bottom picture of FIG. 20 shall be applied at or near or around twelve (12) APs covering BL13×2, BL15×2, BL18×2, BL20×2, and BL23×2 as shown in FIG. 9, DU/GV3 and DU/GV14 as shown in FIG. 16 for the maximum duration of 40 minutes per session;
5.2. for the first 30 minutes of the therapy session, the vacuum cupping pressure shall be maintained approximately around negative 72.5 psi/lb (−5 bar/−0.5 MPa in vacuum) in standard atm pressures, then increase another 14.5 psi/lb (−1 bar/−0.1 MPa) or more, depending upon the amount of substances sucked out, to maintain around negative 87 psi/lb (−6 bar/−0.6 MPa in vacuum) or more in standard atm pressures for the remaining 10 minutes of the therapy session, to achieve an optimal outcome of the lymphatic and blood circulation systems detoxification and cleansing, whereas a clinical standard therapy hygiene practice shall be strictly maintained to treat and dispose all epidermal substances sucked out throughout the session;
5.3. two consecutive sessions shall be performed exactly the same within one day as described in procedures 5.1 and 5.2, whereas the consecutive session shall begin only after the patient have rested for a minimum 90 minutes from the previous session, while taking out any empty cups at the end of each session.
6. Then to start the second batch of vacuum cupping therapy sessions three (3) times a day consecutively for 5 days to follow the procedures below:
6.1. to apply the exact same vacuum cupping therapy sessions as described in procedure 5.1, excluding the empty cups after procedure 5;
6.2. for the first 30 minutes of the therapy session, the vacuum cupping pressure shall be maintained approximately around negative 58 psi/lb (−4 bar/−0.4 MPa in vacuum) in standard atm pressures, then increase another 14.5 psi/lb (−1 bar/−0.1 MPa) or more, depending upon the amount of substances sucked out, to maintain around negative 72.5 psi/lb (−5 bar/−0.5 MPa in vacuum) or more in standard atm pressures for the remaining 10 minutes of the therapy session, to achieve an optimal outcome of the lymphatic and blood circulation systems detoxification and cleansing, whereas a clinical standard therapy hygiene practice shall be strictly maintained to treat and dispose all epidermal substances sucked out throughout the session;
6.3. two consecutive sessions shall be performed exactly the same within one day as described in procedures 6.1 and 6.2, whereas the consecutive session shall begin only after the patient have rested for a minimum 90 minutes from the previous session, while taking out any empty cups at the end of each session.
7. Then to start the last batch of vacuum cupping therapy sessions three (3) times a day consecutively until no more cups remaining by following the procedures below:
7.1. to apply the exact same vacuum cupping therapy sessions as described in procedure 5.1, excluding the empty cups after procedure 6;
7.2. for the first 30 minutes of the therapy session, the vacuum cupping pressure shall be maintained approximately around negative 43.5 psi/lb (−3 bar/−0.3 MPa in vacuum) in standard atm pressures, then increase another 14.5 psi/lb (−1 bar/−0.1 MPa) or more, depending upon the amount of substances sucked out, to maintain around negative 58 psi/lb (−4 bar/−0.4 MPa in vacuum) or more in standard atm pressures for the remaining 10 minutes of the therapy session, to achieve an optimal outcome of the lymphatic and blood circulation systems detoxification and cleansing, whereas a clinical standard therapy hygiene practice shall be strictly maintained to treat and dispose all epidermal substances sucked out throughout the session;
7.3. two consecutive sessions shall be performed exactly the same within one day as described in procedures 7.1 and 7.2, whereas the consecutive session shall begin only after the patient have rested for a minimum 90 minutes from the previous session, while taking out any empty cups at the end of each session.
8. Within 24 hours of completing procedure 7, a final set of preferred aromatic herbal pastes shall be applied at, near or around MGs: DU/GV14, REN/CV8, and DU/GV4 for 3 hours up to 24 hours to calm, heal and close Meridian channels: LU, SP, HT, SI, BL, KD/KID, GB and LV/LIV as shown in FIGS. 3, 6, 7, 8, 9, 10, 13 and 14, whereas the metabolism and homeostasis of a human body is resetting, and the immune response system will be recalled, revitalized and regenerated after the completion of procedure 8.

By completing procedures 1 to 8, the example therapy treatment for detoxification and cleansing of the lymphatic and blood circulation systems of a human body shall demonstrate that a natural alternative way of rebuilding the immune system, revitalize the metabolism, and reset the homeostasis of a human body can be effectively, efficiently, and wholistically realized.

### REFERENCES

Jennifer C. Brookes, Filio Hartoutsiou, A. P. Horsfield, and A. M. Stoneham, “Could Humans Recognize Odor by Phonon Assisted Tunneling?”, Physical Review Letters 98, (2007) 038101.
Xinnong Cheng, Chinese Acupuncture and Moxibustion, 4th edition, October 2019, Foreign Language Press, Beijing China.
John R. Cross, Acupuncture and the Chakra Energy System: Treating the Cause of Disease, 2008, North Atlantic Books, 2526 Martin Luther King Jr. Way, Berkeley, Calif. 94704 USA.
Maria Isabel Francoa, Luca Turina, Andreas Mershin, and Efthimios M. C. Skoulakisa, “Molecular vibration-sensing component in Drosophila melanogaster olfaction”, PNAS 108 (2011) p 3797-3802.
Duo Gao, Chinese medicine, 1997, Thunder's Mouth Press, 245 West 17th Street, New York, N.Y. 10011-5300 USA.
Michio Kaku, The God Equation: The Quest for a Theory of Everything, 2021, Random House Large Print, Random House Tower, 1739 Broadway/230 West 56th Street, New York, N.Y. 10019 USA.
Wynn Kapit & Lawrence M. Elson, The Anatomy Coloring Book, 4th edition, 2019, Pearson Education, Inc., 1900 E. Lake Avenue, Glenview, Ill. 60025 USA.
Seymour Koblin, The Art of Zen-Touch, 4th edition, 2001, Soulstar Creations, 1001 Garnet Avenue #200, San Diego, Calif. 92109 USA.
Henry C. Lu, Traditional Chinese Medicine: How to Maintain Your Health and Treat Illness, 2005, Basic Health Publications, Inc., 28812 Top of the World Drive, Laguna Beach, Calif. 92651 USA.
Susan G. Salvo, Massage Therapy: Principles and Practice, 6th edition, 2020, Elsevier, Inc., 3251 Riverport Lane, St. Louis, Mo. 63043 USA.
Kay W. Sieg & Sandra P. Adams, Illustrated Essentials of Musculoskeletal Anatomy, 6th edition, 2019, Megabooks, Inc., 4111 NW 16th Blvd. #358659, PO Box 358659, Gainesville, Fla. 32635-8659 USA.
Gerard J. Torora & Bryan Derrickson, Principles of Anatomy and Physiology, 13th edition, 2012, John Wiley & Sons, Inc., 111 River Street, Hoboken, N.J. 07030-5774 USA.
Luca Turin, “A Spectroscopic Mechanism for Primary Olfactory Reception”, Chemical Senses 21 (1996) p 773-p 791.
Luca Turin, “The Science of Scent”, TED Talks (2005).
Zhongchao Wu, A Practical Guide to Cupping Therapy: A Natural Approach to Heal through Traditional Chinese Medicine, translated by Jianxin Cao, 2017, Better Link Press, Shanghai Press and Publishing Development Company.

With background theories and knowledge referred in reference section, and core terminologies defined or claimed in definition section, the present invention claimed is:

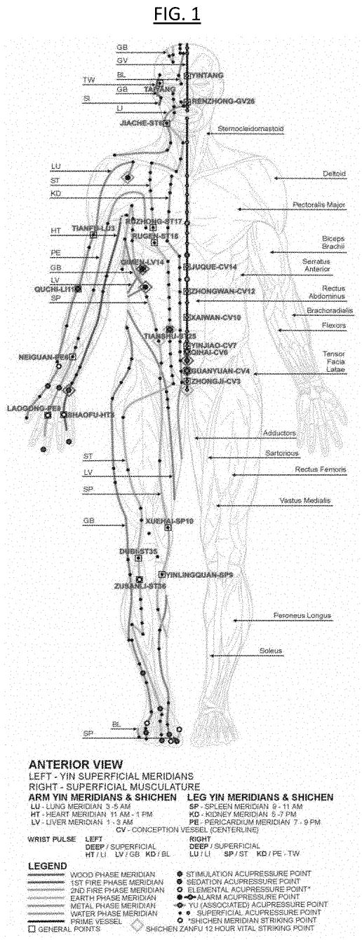

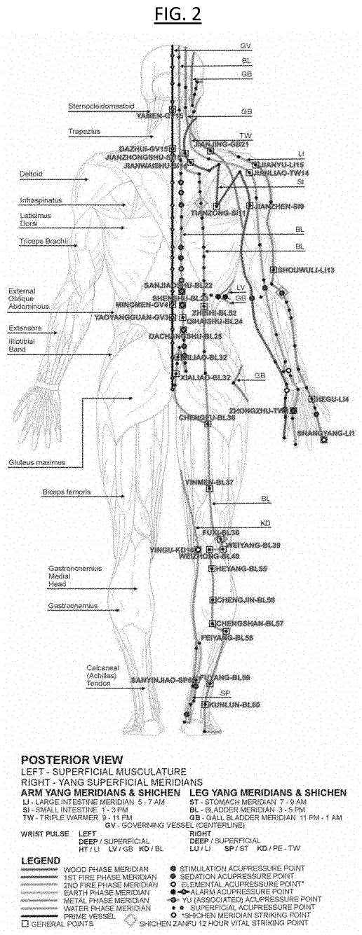

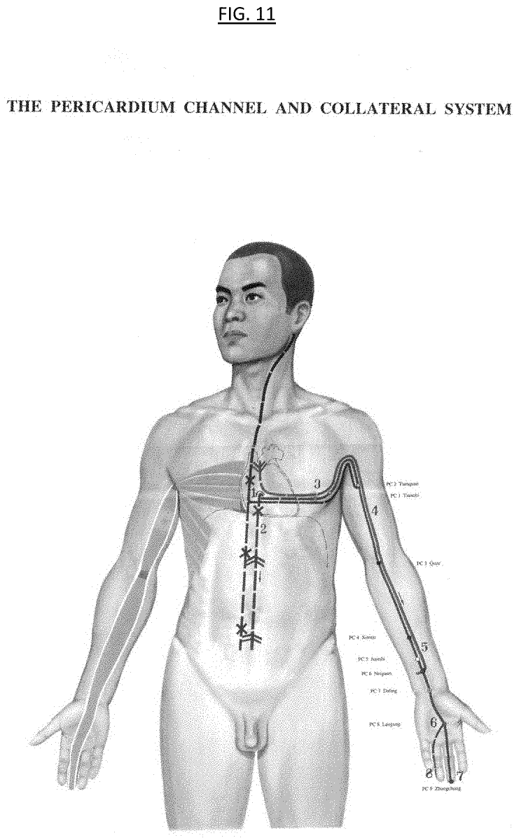

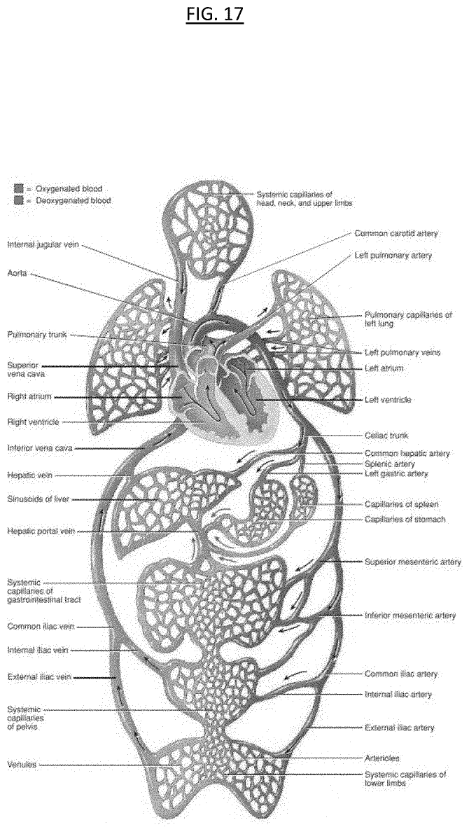

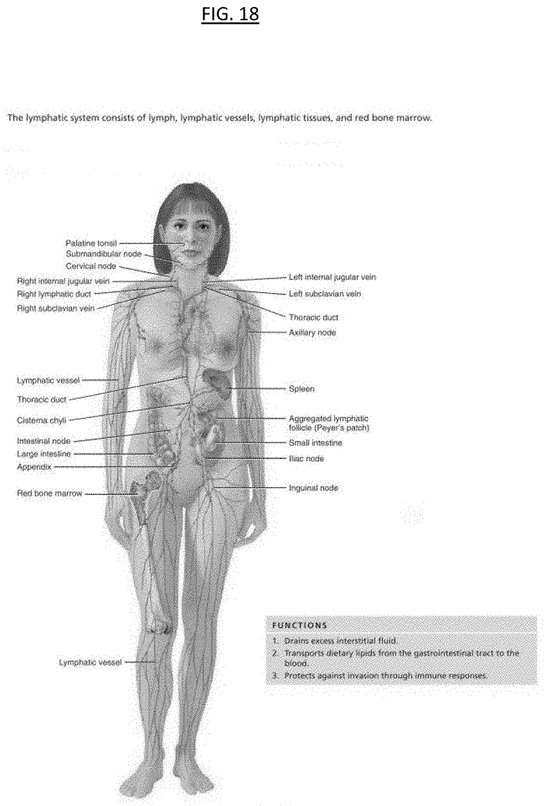

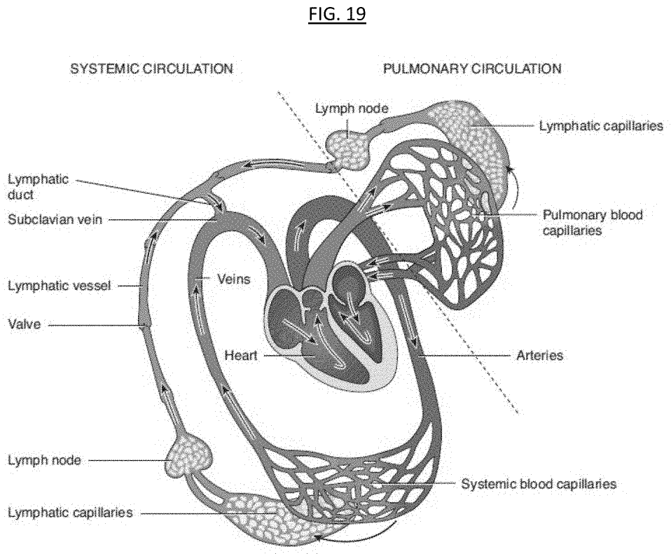

1. A proprietary therapeutic treatment methods, techniques, procedures, and processes to be applied to detox and cleanse the lymphatic and blood circulation systems by a healthcare therapy practitioner comprises:
1.1. at least one set of preferred proprietary acupressure massage techniques selected from the ancient massage methods of Chinese TuiNa, including but not limited to, Tui, Na, An, Mo, Tien, Mot, Tsa, Pok, Cha, Niem or Japanese Shiatsu, and based on traditional knowledges and empirical experiences of Meridian theory will be applied to enable, activate and drive quantum Chi energy on at least one of fourteen (14) Meridian channels as illustrated from FIG. 3 to FIG. 16;
1.2. at least three subsets of 670 APs as shown in FIG. 1-2, composed of at least two (2) MGs per subset selected by their empirical functional efficacies known and experienced in ancient Meridian theory, will be performed at, near or around by a therapy practitioner per description in claim 1.1, to attain at least three preferred functional efficacies outcomes corresponding to 1) energizing, and opening; 2) transforming, and transporting; and 3) healing, and closing respectively, where each MGs subset will relate to at least one preferred Meridian channel to be controlled;
1.3. at least one set of negative energy force by means like the traditional cupping therapy as shown in top picture of FIG. 20 will be applied epidermically at, near or around at least two (2) APs of one preferred Meridian channel controlled by MGs in claim 1.2 to further enhance their functional efficacies;
1.4. at least two sets of quantum energy means shall be administered or applied at, near or around MGs in claim 1.2 to have their functional efficacies optimized for opening or for transporting respectively, whereas their preferred Meridian channels will then be affected, opened and enabled to transport for the therapy treatment of their deeply associated lymphatic and cardiovascular blood circulation systems as shown in FIG. 17-19;
1.5. at least one set of negative energy force or means like using clinically controlled and professionally managed vacuum cupping therapy session as shown in bottom picture of FIG. 20 will be applied epidermically, systematically, and consecutively at, near or around at least two (2) APs of one preferred Meridian channel practiced in claim 1.2 at least three (3) times in 24 hours, which the negative energy force or means will be approximately controlled in terms of vacuum atmosphere pressure within an empirically experienced therapy duration, to effectively, quantitatively and therapeutically accomplish and achieve the detoxification and cleansing of the lymphatic and blood circulation systems on a human body;
1.6. at least one set of proprietary means of quantum energy shall be administered or applied to have the optimized functional efficacies of healing and closing corresponded to the functional efficacies of the MGs subsets as described in claim 1.2.

2. For every therapeutic treatment to detox and cleanse the lymphatic and blood circulation systems by a healthcare therapy practitioner, the invention claim 2 comprises:
2.1. at least one set of preferred acupressure massage techniques will be applied at, near or around two (2) MPs for controlling one preferred Meridian channel to best attain the desired functional efficacies in the shortest time, where quantum Chi movement is proportionally measured with Cun by empirical knowledges and experiences per Meridian theory;
2.2. the maximum duration of each therapy session per treatment shall be determined by considering claim 2.1, and circumstantial factors including, but not limited to, patient's physical conditions, medical data, treatment tools and means;
2.3. the minimum duration to separate each therapy session per treatment shall be determined by guaranteeing the optimal time required to re-establish the homeostasis conditions in a patient after extracting or depleting the bodily substances during the detoxification and cleansing procedures administrated by a healthcare practitioner.

3. For every therapeutic treatment to detox and cleanse the lymphatic and blood circulation systems by a healthcare therapy practitioner, the invention claim 3 comprises at least twelve (12) MPs for effecting and controlling six (6) preferred Meridian channels like LU, SP, HT, BL, KD/KID, and LV/LIV as shown in FIGS. 3, 6, 7, 9, 10, and 14 to be applied at, near or around by the set of preferred acupressure massage techniques per claim 1.1 and claim 2.1, while all available factors, especially the harmful contraindications, and mutual consent will be considered to initiate a therapy treatment.

4. For every therapeutic treatment to detox and cleanse the lymphatic and blood circulation systems by a healthcare therapy practitioner, the invention claim 4 comprises at least one set of negative energy force by means like using the traditional cupping therapy as shown in top picture of FIG. 20 will be applied epidermically at, near or around at least twelve (12) preferred APs of six (6) preferred Meridian channels per claim 1.3 and claim 3.

5. For every therapeutic treatment to detox and cleanse the lymphatic and blood circulation systems by a healthcare therapy practitioner, the invention claim 5 comprises at least two sets of quantum energy means like two sets of aromatic herbal scent pastes shall be administered or applied at, near or around at least ten (10) MGs that will have their functional efficacies optimized for opening or for transporting respectively to effect and control the six (6) preferred Meridian channels per claim 3.

6. For every therapeutic treatment to detox and cleanse the lymphatic and blood circulation systems by a healthcare therapy practitioner, the invention claim 6 comprises:
6.1. at least one set of negative energy force or means like using clinically controlled and professionally managed vacuum cupping therapy session as shown in bottom picture of FIG. 20 will be applied epidermically, systematically, and consecutively at, near or around at least twelve (12) APs of six (6) preferred Meridian channels per claim 1.5 and claim 4 at least three (3) times in 24 hours, which the negative energy force or means will be applied by increasing, reducing and maintaining an optimal vacuum atmosphere pressure per empirical knowledge of Meridian theory to effectively and therapeutically extract out the substances per therapy session for best achieving the detoxification and cleansing results per treatment according to session duration in claim 2.2 and session separation in claim 2.3, until no more substances being extracted out epidermically by the negative energy force or means.
6.2. a set of therapy session in a treatment applying negative energy force or means with the optimal vacuum atmosphere pressure settings per claim 6.1 shall be performed at least consecutively for seven (7) days before adjusting to new pressure settings in a session thereafter, which a strict clinical therapy hygiene standard will be maintained and practiced to treat and dispose the epidermal substances extracted or sucked out through the epithelial tissues during the whole treatment.

7. For every therapeutic treatment to detox and cleanse the lymphatic and blood circulation systems by a healthcare therapy practitioner, the invention claim 7 comprises at least one set of quantum energy means like using a set of aromatic herbal scent pastes shall be administered or applied at, near or around at least two (2) MGs that will have the optimized functional efficacies of healing and closing for effecting and controlling the six (6) preferred Meridian channels per claim 3.

8. With every therapeutic treatment to detox and cleanse the lymphatic and blood circulation systems by a healthcare therapy practitioner, the invention claim 8 shall guarantee that the metabolism and homeostasis of a human body can be reset to a new start, it will demonstrate that by cleaning up the cesspools of a human body, this therapeutic treatment can provide a powerful and natural way to rebuild and protect a human immune system where it can provide an effective alternative means to treat and cure many unforeseen diseases and illness including, but not limited to, clotting, thrombus, embolus, cerebral infarction, and stroke;

9. For every therapeutic treatment to detox and cleanse the lymphatic and blood circulation systems by a healthcare therapy practitioner, the invention claim 9 shall treat and regard the pains, which are caused by applying the negative energy force or means of using the vacuum cupping to extract and suck out the substances through the epithelial tissues during each therapy session, as a part of the treatment to recall, revitalize and regenerate the immune response system of a human body.
